(19) United States
(12) Patent Application Publication
(10) Pub. No.: US 2026/0165635 A1
(43) Pub. Date: Jun. 18, 2026

(54) PLANTAR NEUROPATHY TEST DEVICE

(71) Applicant: Allison Lievano, Garches (FR)

(72) Inventor: Allison Lievano, Garches (FR)

(21) Appl. No.: 19/358,222

(22) Filed: Oct. 14, 2025

Related U.S. Application Data
(60) Provisional application No. 63/707,703, filed on Oct. 15, 2024.

(51) Int. Cl. A61B 5/00; A61B 90/00

(52) U.S. Cl. CPC A61B 5/4041; A61B 5/0053; A61B 90/06; A61B 90/08; A61B 2090/064; A61B 2090/0807; A61B 2560/0223; A61B 2562/12

(57) ABSTRACT

Systems and methods as described herein relate to a neuropathy detection instrument. The instrument can include a cylindrical casing. A piston with a rod can be disposed within the cavity, and the rod can be configured to fit within an opening to be pressed against the skin of a patient. A helical compression spring disposed adjacent to the rod and disposed between the piston and the casing. The helical compression spring can compress against the casing as pressure is applied to the rod and the rod and piston move along the cavity. The compression can be tracked against a target compression to determine a likelihood of whether neuropathy is present.

### CROSS-REFERENCE TO RELATED APPLICATION(S)

The present application claims priority to U.S. Provisional Patent Application No. 63/707,703, titled “PLANTAR NEUROPATHY TEST DEVICE” and filed Oct. 15, 2024, the entirety of which is incorporated by reference herein.

### FIELD

The present application generally relates to devices and methods to screen for loss in sensation in a patient, such as at a sole of a foot of a patient to screen for plantar neuropathy.

### BACKGROUND

Neuropathy, such as plantar neuropathy, is a condition where nerves on the human skin, such as at the bottom of the foot, become diseased. This condition often causes weakness, numbness and pain, usually in the hands and feet. Neuropathy can be caused by various health conditions such as autoimmune diseases, diabetes, infections, etc.

Detection and screening for neuropathy can be performed via any of diagnostic tests, examinations, blood testing, studies, etc. Particularly, instruments can be used to detect neuropathy. For instance, an instrument can use a polymer monofilament that is pressed against the skin of a patient that can buckle or bend suddenly at a specific critical force. Such a mechanism can allow for a standardized test to be performed, applying a repeatable force and pressure to the skin of the user.

However, such instruments may present several issues. For example, accuracy can be limited because due to natural variability in polymer elasticity and varying manufacturing specifications, the actual force at which the monofilament buckle (and thus the pressure applied to the patient's skin) often deviates, sometimes significantly, from the labeled buckling force from one instrument to another. Further, such instruments can have limited precision in that the buckling force can vary significantly from one application of the instrument to another, often exceeding the desired precision of ±10% variability. Additionally, performance deterioration with cyclic loading can be present in that a reduction in the buckling force due to repeated use (cyclic loading) can be consistently reported in the literature, following a logarithmic decay. After only a few hundred cycles, the buckling force can deviate by more than 10% from its initial value. Further, such instruments can have temperature sensitivity in that the buckling force can be affected by temperature, which can compromise the test's performance in hot weather.

Accordingly, there is a need for a detection instrument that provides for accurate and consistent detection of neuropathy.

### SUMMARY

Embodiments of the present disclosure may include systems and methods relating to a neuropathy detection instrument. The instrument can include a cylindrical casing. A piston with a rod can be disposed within the cavity, and the rod can be configured to fit within an opening to be pressed against the skin of a patient. A helical compression spring disposed adjacent to the rod and disposed between the piston and the casing. The helical compression spring can compress against the casing as pressure is applied to the rod and the rod and piston move along the cavity. The compression can be tracked against a target compression to determine a likelihood of whether neuropathy is present.

In an example embodiment, a plantar neuropathy detection instrument comprises a cylindrical casing. A cavity can be formed in the cylindrical casing. The casing can include an opening can be formed in a center portion at a first end of the casing.

The instrument can include a piston disposed within the cavity and a rod connected to the piston. The rod is configured to move through the opening. The instrument can also include a helical compression spring disposed adjacent to the rod and disposed between the piston and the casing. The helical compression spring can be configured to compress against the casing as pressure is applied to the rod and the rod and piston move along the cavity.

In some instances, the rod has a diameter of around 0.6 millimeters (mm).

In some instances, the rod is configured to be pressed perpendicular to a skin of a subject and partially retract into the casing.

In some instances, the helical compression spring is comprised of a stainless-steel material.

In some instances, the instrument can include a slit formed in a side of the casing. In some instances, the slit comprises a magnifying plastic or glass material to view the piston within the cavity through the slit.

In some instances, the instrument can include a set of scale marking disposed on an exterior of the casing where the slit is formed. In some instances, the instrument can also include an adjustable marker disposed on the exterior of the casing. The adjustable marker can be configured to be moved along an exterior of the casing for calibration of movement of the piston.

In some instances, a target compression of the rod and piston when applied to a patient is determined by correlating a force applied to the rod with a compression distance of the spring. In some instances, a target compression of the spring is around 8 mm.

In another example embodiment, a device is provided. The device can include a casing. An opening can be formed in a center portion at a first end of the casing. The device can include a piston disposed within a cavity formed in the casing and a rod connected to the piston. The rod can be configured to move through the opening. The device can also include a spring disposed between the piston and the casing.

In some instances, the spring is configured to compress against the casing as pressure is applied to the rod and the rod and piston move along the cavity.

In some instances, the rod is configured to be pressed perpendicular to a skin of a subject and partially retract into the casing.

In some instances, the device comprises a slit formed in a side of the casing. In some instances, the slit comprises a magnifying plastic or glass material to view the piston within the cavity through the slit.

In some instances, the device can include a set of scale marking disposed on an exterior of the casing where the slit is formed and an adjustable marker disposed on the exterior of the casing. The adjustable marker can be configured to be moved along an exterior of the casing for calibration of movement of the piston. In some instances, a target compression of the rod and piston when applied to a patient is determined by correlating a force applied to the rod with a compression distance of the spring.

In some instances, the device can include a first set of tabs formed on a first half of the casing and a second set of tabs formed on a second half of the casing, wherein a fastener is configured to be disposed through an opening of each of a first tab of the first set of tabs and the second set of tabs and a second tab of the first set of tabs and the second set of tabs to secure the first half of the casing to the second half of the casing.

In another example embodiment, a method for manufacturing a neuropathy detection instrument can include providing a cylindrical casing. A cavity can be formed in the cylindrical casing. The method can also forming an opening in a center portion at a first end of the casing.

The method can also include disposing a piston disposed within the cavity. A rod can be connected to the piston, The rod can be configured to move through the opening. The method can also include disposing a helical compression spring adjacent to the rod and between the piston and the casing. The helical compression spring can be configured to compress against the casing as pressure is applied to the rod and the rod and piston move along the cavity.

In some instances, the method can also include disposing a set of scale marking on an exterior of the casing where a slit is formed in the casing and disposing an adjustable marker disposed on the exterior of the casing. The adjustable marker can be configured to be moved along an exterior of the casing for calibration of movement of the piston.

In some instances, a target compression of the rod and piston when applied to a patient is determined by correlating a force applied to the rod with a compression distance of the spring.

### BRIEF DESCRIPTIONS OF THE DRAWINGS

For a better understanding of the embodiments described in this application, reference should be made to the Detailed Description below, in conjunction with the following drawings in which like reference numerals refer to corresponding parts throughout the figures.

FIG. 1 is a view of a portion of an example neuropathy detection instrument in accordance with certain aspects described herein;

FIG. 2 illustrates an exterior of a portion of an example instrument in accordance with certain aspects described herein;

FIG. 3 is a top view of a portion of an example instrument in accordance with certain aspects described herein;

FIG. 4 is a side view of a portion of an example instrument in accordance with certain aspects described herein;

FIG. 5 is a second side view of a portion of an example instrument in accordance with certain aspects described herein;

FIG. 6 is a third side view of a portion of an example instrument in accordance with certain aspects described herein;

FIG. 7 illustrates an example instrument in accordance with certain aspects described herein;

FIG. 8 illustrates a side view of an example instrument in accordance with certain aspects described herein;

FIGS. 9A-9C illustrate views of an example second half of an instrument in accordance with certain aspects described herein;

FIGS. 10A-C illustrate various side views of the second half of the instrument in accordance with certain aspects described herein;

FIGS. 11A-E illustrate views of an example push plate in accordance with certain aspects described herein;

### DETAILED DESCRIPTION

Reference will now be made in detail to embodiments, examples of which are illustrated in the accompanying drawings. In the following detailed description, numerous specific details are set forth in order to provide a sufficient understanding of the subject matter presented herein. But it will be apparent to one of ordinary skill in the art that the subject matter may be practiced without these specific details. Moreover, the particular embodiments described herein are provided by way of example and should not be used to limit the scope of the particular embodiments.

The present embodiments generally relate to devices and methods for detecting a loss of sensation in a sole of the foot (e.g., indicative of plantar neuropathy) or in other areas of the skin of a patient. While the example of detecting plantar neuropathy in the sole of the foot of a patient, the embodiments are not generally limited to such examples. For instance, the instrument as described herein can detect various nerve-related conditions at various places on the human body. As another example, the instrument can be used as an aesthesiometer for measuring the tactile sensitivity of the skin.

Other instrument designs may use a polymer monofilament that is pressed against the skin that can buckle or bend suddenly at a specific critical force. Such a mechanism can allow for a standardized test to be performed, applying a repeatable force and pressure to the skin of the user.

However, such instruments may present several issues. For example, accuracy can be limited because due to natural variability in polymer elasticity and varying manufacturing specifications, the actual force at which the monofilament buckle (and thus the pressure applied to the patient's skin) often deviates, sometimes significantly, from the labeled buckling force from one instrument to another. Further, such instruments can have limited precision in that the buckling force can vary significantly from one application of the instrument to another, often exceeding the desired precision of ±10% variability. Additionally, performance deterioration with cyclic loading can be present in that a reduction in the buckling force due to repeated use (cyclic loading) has be consistently reported in the literature, following a logarithmic decay. After only a few hundred cycles, the buckling force can deviate by more than 10% from its initial value. Further, such instruments can have temperature sensitivity in that the buckling force can be affected by temperature, which can compromise the test's performance in hot weather.

The present embodiments can provide a screening instrument having an improved actuating mechanism of a simple and reliable character that can require fewer resources to produce. The instrument as described herein to screen for loss of sensation on human skin can include a cylindrical casing that houses a slender, rigid rod. The casing can be made from a range of suitable materials, including materials such as 3D-printable polymers, metals, or other durable materials. Since the device does not penetrate the skin, biocompatibility concerns are minimal.

The compression of the instrument used against the skin of a patient can be tracked against a target compression. Further, the compression of the instrument can be compared against whether the patient indicates a feeling of the device being pressed against the skin, which can be indicative of whether neuropathy or a similar condition is present.

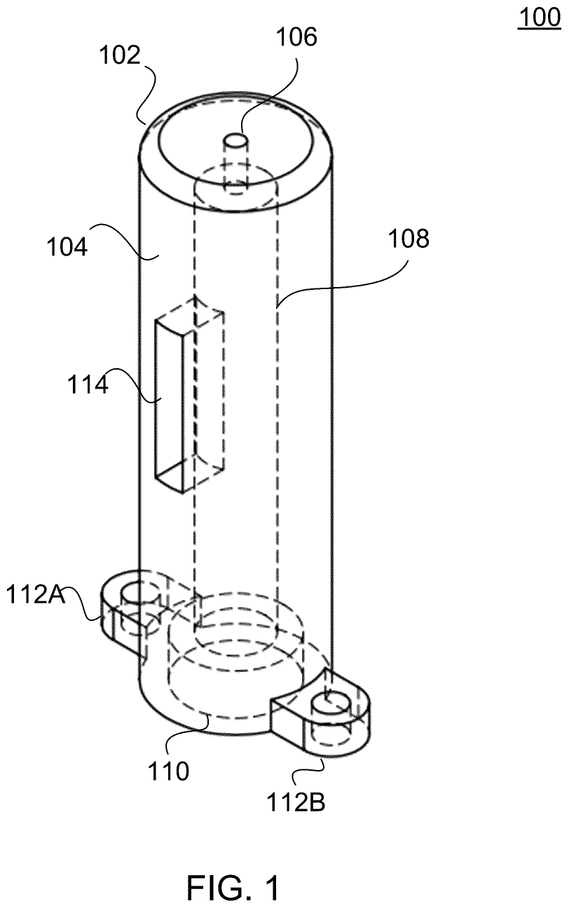

FIG. 1 is a view of a part of an example neuropathy detection instrument 100. The instrument 100 can include an outer casing 102 that comprises a cylindrical shape. The casing 102 can include a cavity 104 that houses a rod 106 and piston 108.

The rod 106 can be approximately 0.6 mm in diameter to match the size used in monofilament-based devices, ensures consistent pressure application to the skin. This diameter can help the device not only match the force but also replicate the pressure delivered by standard devices. The pressure applied can be calculated to be significantly below the threshold required to pierce the skin, ensuring safety during use. The rod 106 can be made of a material such as stainless steel or other suitable metals, or various high-strength composite materials known for stiffness and corrosion resistance.

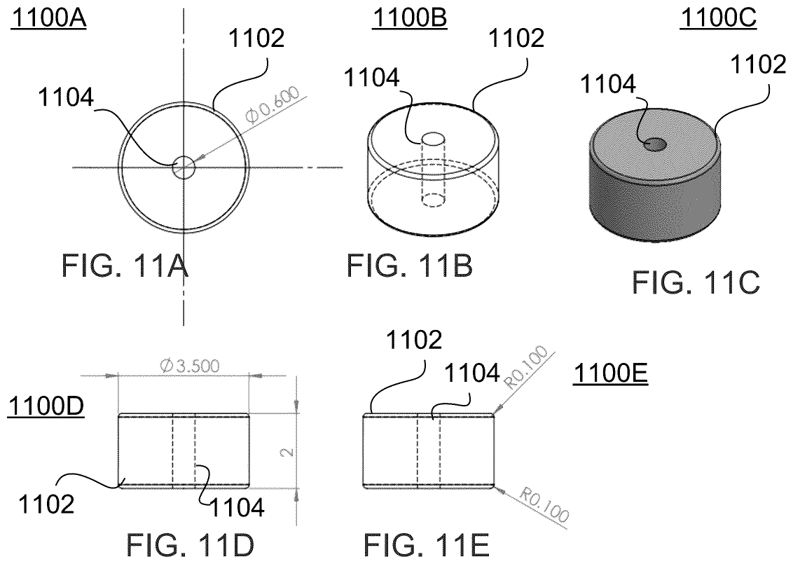

A push plate 110 can be disposed adjacent to the piston 108. The push plate 110 can be used to actuate the piston 108 and rod 106 as described herein. The push plate is described in greater detail with respect to FIGS. 11A-11E.

A helical compression spring can surround the rod and can be positioned between the piston and an internal wall of the casing. The internal wall can include a circular orifice through which the rod can pass but not the spring, effectively anchoring one end of the spring. The spring can be made of stainless steel or music wire, both of which can provide sufficient elasticity and durability. As pressure is applied to the rod, both the rod and the piston move together, compressing the spring against the internal wall.

The instrument 100 can also include tabs 112A, 112B that extend outwardly from the casing 102. The tabs 112A-B can allow for connecting a first half of the instrument (e.g., portion 732A) to a second half of the instrument (e.g., portion 732B) using respective tabs (e.g., 828A, 828B) on the second half of the instrument. The tabs can include openings that allow a screw or other fastener to connect the instrument to another portion of the instrument.

The instrument 100 can also include a slit 114 formed in the casing 102. The slit 114 can allow for viewing of the piston 108 through the casing 102. The slit can be substantially rectangular in shape, or comprising another shape with a length larger than its width.

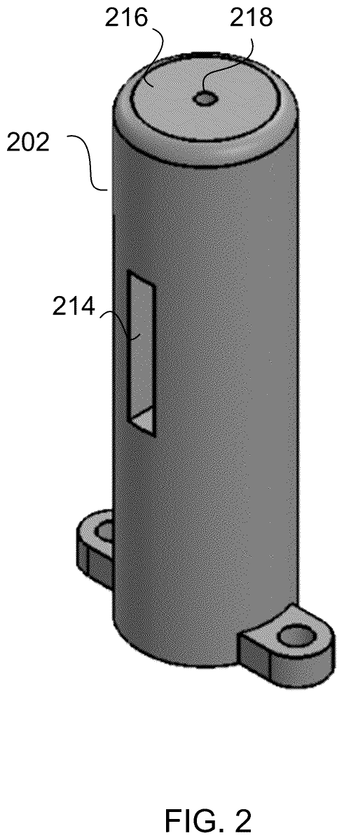

FIG. 2 illustrates an exterior of a portion of an example instrument 200. As shown in FIG. 2, the outer casing 202 can include slit 214. The first end 216 of the instrument can include an opening 218 or center portion that can allow the rod to protrude and move back and forth.

The portion of the rod protruding from the uncompressed device can be only a few millimeters greater than the target compression distance. This design can ensure that if excessive force is applied, the rod can fully retract into the casing, preventing any risk of skin penetration.

When the rod is pressed perpendicularly against the skin, it can gradually and partially retract into the casing. Inside the casing, the rod can be securely attached to a piston. The piston can be made from 3D-printable polymers or, coated or constructed with very low-friction materials like Polytetrafluoroethylene (PTFE) or TEFLON to ensure smooth movement and reduce friction.

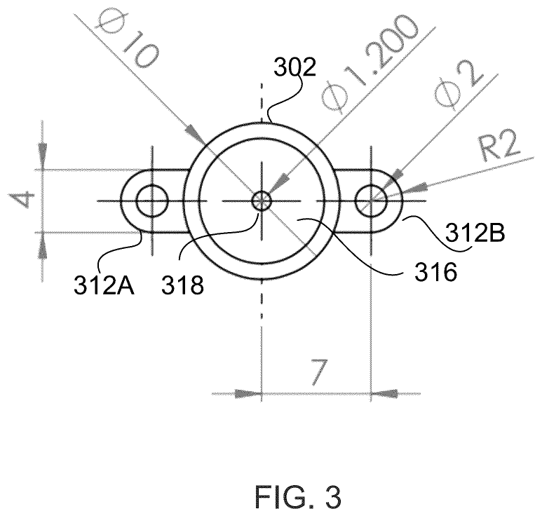

FIG. 3 is a top view of an example instrument 300. As shown in FIG. 3, the first end 316 of the casing 302 can have a diameter of around 10 mm or ranging between 5-15 mm, for example, while the opening 318 can have a diameter of around 1.2 mm. The tabs 312A, 312B can include an opening with a diameter of around 2 mm and a width of around 4 mm.

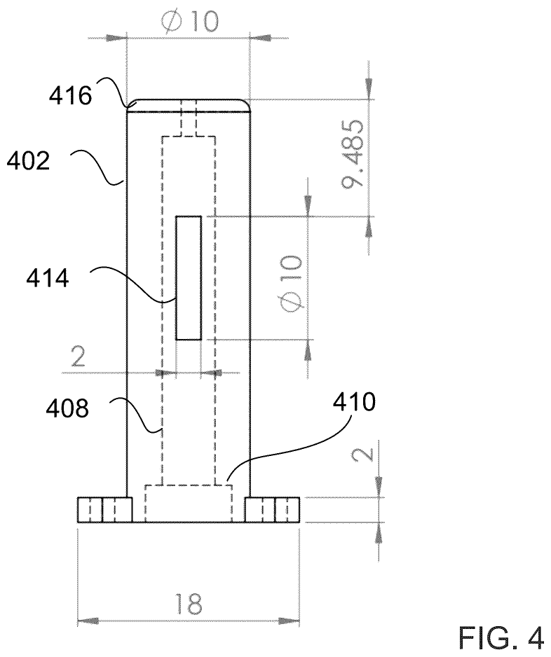

FIG. 4 is a side view of a portion of an example instrument 400. As shown in FIG. 4, the casing 402 can have a diameter of around 10 mm. The instrument 400 can also include a piston 408 and push plate 410. A distance from a top of a first end 416 to a beginning of a slit 414 can be around 9.485 mm, while a length of the slit 414 can be around 10 mm and the width of the slit 414 can be around 2 mm.

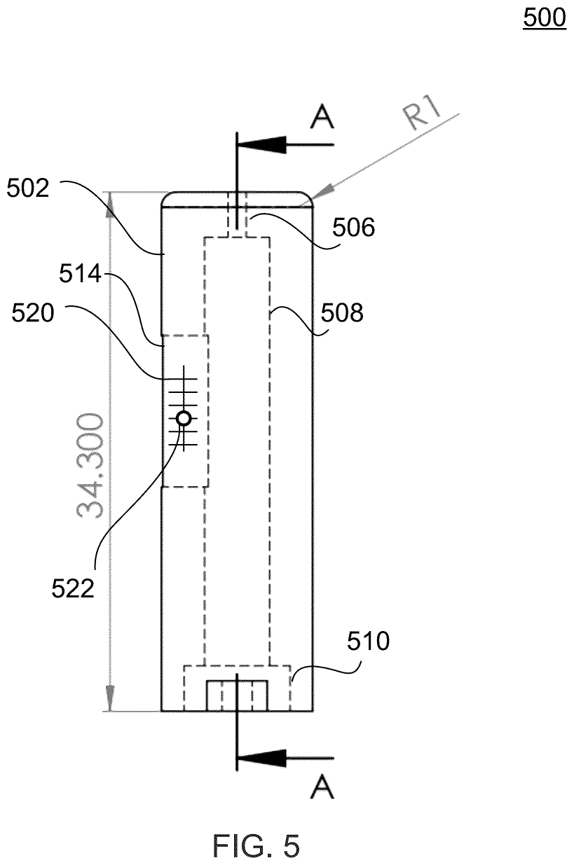

FIG. 5 is a second side view of a portion of an example instrument 500. As shown in FIG. 5, the casing 502 can form a slit 514 covered with magnifying plastic or glass to enhance the monitoring of the piston's movement. The piston 508 can be of a contrasting color to the casing 502, and scale markings 520 are placed alongside the slit, providing precise measurements of the piston's displacement. This setup can allow the operator to correlate the compression distance directly with the applied force.

An adjustable marker 522 can be positioned on the exterior of the casing 502 adjacent to scale markings 520. This marker can indicate when the target force has been reached and can be moved to recalibrate the device. Calibration can include adjusting the marker to correspond with the desired compression distance that equates to the target force, accommodating normal variability in the spring constant from one device to another or changes due to cyclical loading of the spring.

The instrument can measure the force applied to the skin by correlating it with the compression distance of the spring, visible through the magnified slit and quantified by the scale markings. In practice, there can be a target force corresponding to a specific compression of the spring in millimeters. It can be important that this target compression be greater than 8 mm. Experimental data indicates that the absolute error in manual operation remains substantially constant across different compression magnitudes and that maintaining a minimum compression of at least 8 mm ensures that the relative error stays under 10%.

Additionally, the target compression can fall within the linear range of the spring—the compression range over which the spring follows Hooke's Law, exhibiting a linear proportional relationship between compression and force. This typically spans from 20% to 80% of the spring's full compression capacity. Selecting a spring with an appropriate spring constant can be essential to ensure that the target compression (above 8 mm) falls within this linear range.

This instrument provides an effective and safe means to assess sensory loss on the skin. It matches or exceeds the accuracy and precision in delivering both the force and pressure parameters of standard monofilament-based devices while offering much longer resistance to cyclic loading, enhanced usability and safety features.

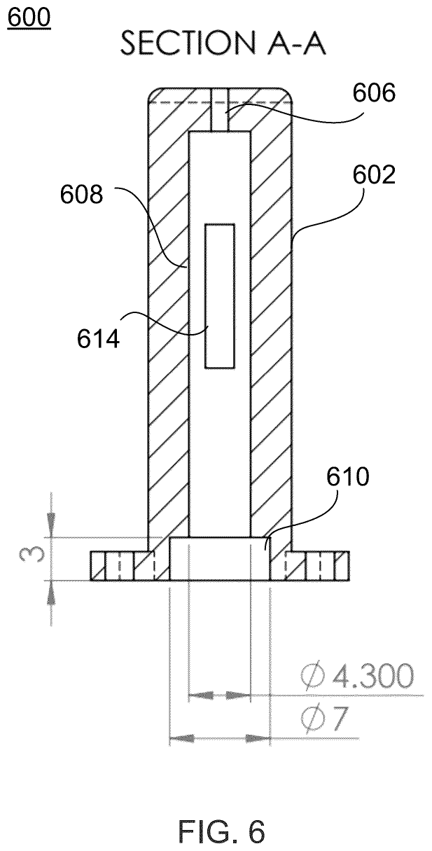

FIG. 6 is a third side view of a portion of an example instrument 600. As shown in FIG. 6, the instrument 600 can include a casing 602, piston 608 and rod 606 formed within a cavity of the casing 602, and a slit 614 formed in the casing 602. The push plate 610 can be disposed between the piston 608 and the casing 602. A width or a diameter of the piston 608 can be around 4.3 mm, while the width/diameter of the push plate 610 can be around 7 mm.

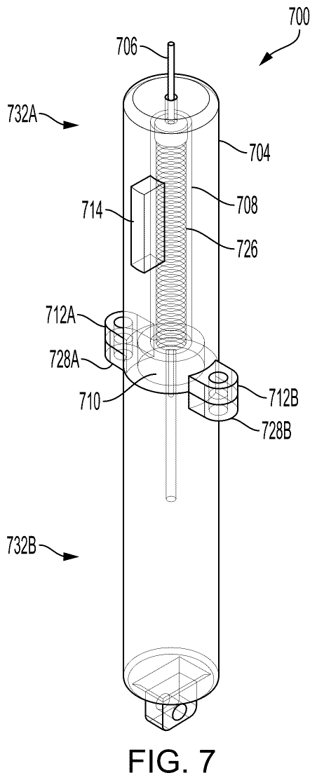

FIG. 7 illustrates an example instrument 700. As shown in FIG. 7, the instrument 700 can include a first half 732A and a second half 732B. The first half 732A can include a casing 704, rod 706, piston 708, and a slit 714. A helical compression spring 726 can be disposed adjacent to the rod 706 and/or the piston 708.

The first half 732A can also include tabs 712A, 712B that are configured to connect to tabs 728A, 728B on the second half 732B. The rod 706 can extend into the second half 732B of the instrument 700.

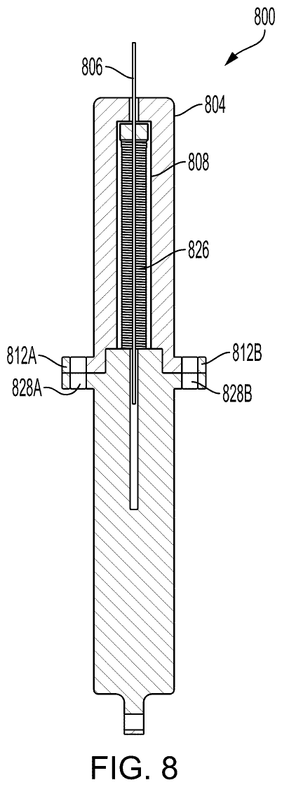

FIG. 8 illustrates a side view of an example instrument 800. As shown in FIG. 8, the instrument 800 can include a casing 804, rod 806, piston 808, a slit 814, and a spring 826. Tabs 812A, 812B can connect to tabs 828A, 828B.

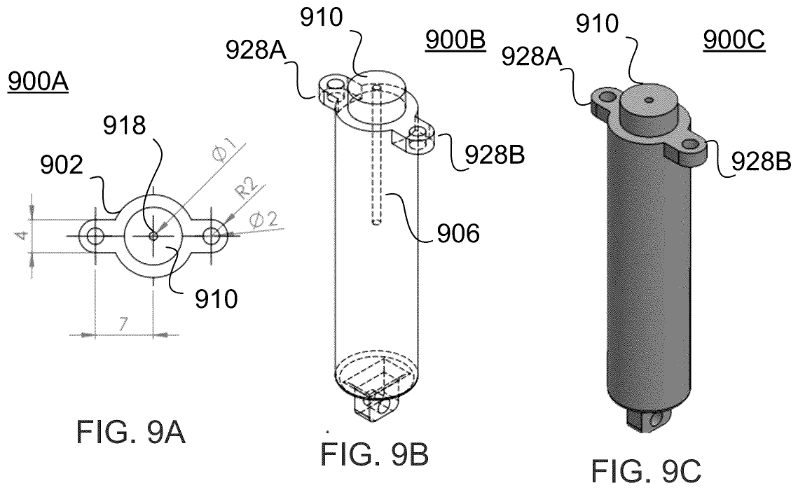

FIGS. 9A-9C illustrate views of an example second half of an instrument. FIG. 9A illustrates a top view 900A of the second half of the instrument, depicting the casing 902 and a push plate 910. An opening 918 can be formed in the push plate 910 to allow a rod 906 to move through the push plate 910.

FIG. 9B is a first side view 900B of the second half of the instrument, showing the tabs 928A, 928B, the push plate 910, and the rod 906. FIG. 9C is a second side view 900C of the second half of the instrument, showing the tabs 928A, 928B and the push plate 910.

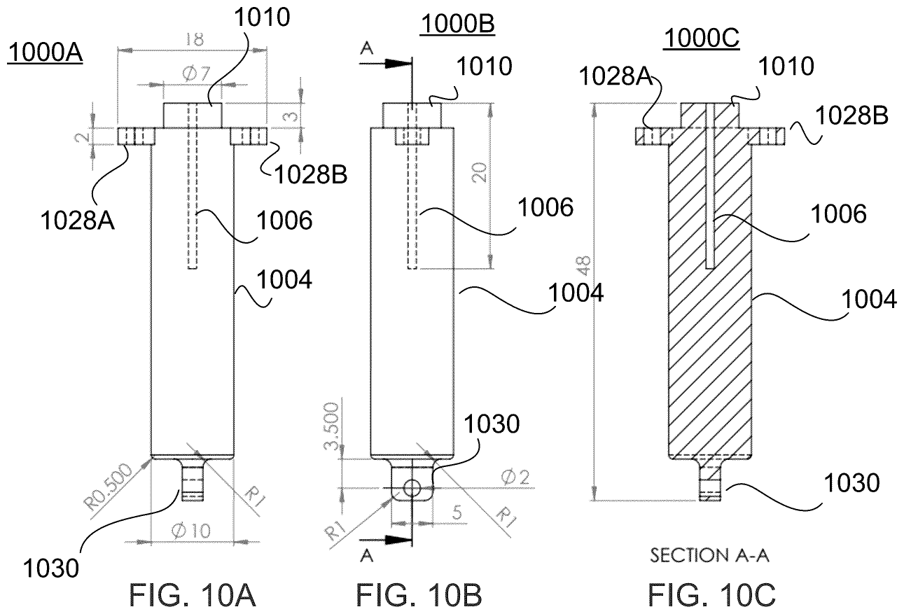

FIGS. 10A-C illustrate various side views 1000A-1000C of the second half of the instrument. The second half 1000A-1000C can include a casing 1004, push plate 1010 and tabs 1028A, 1028B, and rod 1006. The instrument can also include a bottom component 1030. The bottom component 1030 can act as a fixture to receive a strap or string to handle or hang the instrument, such as around the neck of the operator, for example.

In some examples, the instrument can include a cover can fit over the top of the casing to protect the rod when the instrument is not in use.

FIGS. 11A-E illustrate views of an example push plate 1100A-E. The push plate 1100A-E can include an outer housing 1102 and an opening 1104 that allows for movement of the rod. A diameter of the opening 1104 can be around 0.6 mm, a width of the push plate 1100 can be around 3.5 mm, and a height of the push plate 1100 can be around 2 mm.

The foregoing description, for purpose of explanation, has been described with reference to specific embodiments. However, the illustrative discussions above are not intended to be exhaustive or to limit the embodiments to the precise forms disclosed. Many modifications and variations are possible in view of the above teachings. The embodiments were chosen and described in order to best explain the principles of the embodiments and its practical applications, to thereby enable others skilled in the art to best utilize the various embodiments with various modifications as are suited to the particular use contemplated.

What is claimed is:

1. A neuropathy detection instrument comprising:
a cylindrical casing, wherein a cavity is formed in the cylindrical casing, and wherein an opening is formed in a center portion at a first end of the casing;
a piston disposed within the cavity;
a rod connected to the piston, wherein the rod is configured to move through the opening; and
a helical compression spring disposed adjacent to the rod and disposed between the piston and the cylindrical casing, wherein the helical compression spring is configured to compress against the cylindrical casing as pressure is applied to the rod.

2. The neuropathy detection instrument of claim 1, wherein the rod has a diameter of around 0.6 millimeters (mm).

3. The neuropathy detection instrument of claim 1, wherein the rod is configured to be pressed perpendicular to a skin of a subject and partially retract into the cylindrical casing.

4. The neuropathy detection instrument of claim 1, wherein the helical compression spring is comprised of a stainless steel material.

5. The neuropathy detection instrument of claim 1, further comprising:
a slit formed in a side of the cylindrical casing, wherein the slit comprises a magnifying plastic or glass material to view the piston within the cavity through the slit.

6. The neuropathy detection instrument of claim 1, further comprising:
A push plate disposed at an end of the instrument opposite the opening, wherein the push plate is configured to move the piston and rod relative to the casing as pressure is applied to the push plate.

7. The neuropathy detection instrument of claim 5, further comprising:
a set of scale marking disposed on an exterior of the cylindrical casing where the slit is formed.

8. The neuropathy detection instrument of claim 7, further comprising:
an adjustable marker disposed on the exterior of the cylindrical casing, wherein the adjustable marker is configured to be moved along an exterior of the cylindrical casing for calibration of movement of the piston.

9. The neuropathy detection instrument of claim 1, wherein a target compression of the rod and piston when applied to a patient is determined by correlating a force applied to the rod with a compression distance of the helical compression spring.

10. The neuropathy detection instrument of claim 9, wherein a target compression of the helical compression spring is around 8 mm.

11. A device comprising:
a casing, wherein an opening is formed in a center portion in a first end of the casing;
a piston disposed within a cavity formed in the casing;
a rod connected to the piston, wherein the rod is configured to move through the opening; and
a spring disposed between the piston and the casing.

12. The device of claim 11, wherein the spring is configured to compress against the casing as pressure is applied to the rod, and the rod and the piston move along the cavity.

13. The device of claim 11, wherein the rod is configured to be pressed perpendicular to a skin of a subject and partially retract into the casing.

14. The device of claim 11, further comprising:
a slit formed in a side of the casing.

15. The device of claim 14, wherein the slit comprises a magnifying plastic or glass material to view the piston within the cavity through the slit.

16. The device of claim 15, further comprising:
a set of scale marking disposed on an exterior of the casing where the slit is formed; and
an adjustable marker disposed on the exterior of the casing, wherein the adjustable marker is configured to be moved along an exterior of the casing for calibration of movement of the piston.

17. The device of claim 11, further comprising:
a first set of tabs formed on a first half of the casing; and
a second set of tabs formed on a second half of the casing, wherein a fastener is configured to be disposed through an opening of each of a first tab of the first set of tabs and the second set of tabs and a second tab of the first set of tabs and the second set of tabs to secure the first half of the casing to the second half of the casing.

18. A method for manufacturing a neuropathy detection instrument, the method comprising:
providing a cylindrical casing, wherein a cavity is formed in the cylindrical casing;
forming an opening in a center portion at a first end of the cylindrical casing;
disposing a piston disposed within the cavity, wherein a rod is connected to the piston, and wherein the rod is configured to move through the opening; and
disposing a helical compression spring adjacent to the rod and between the piston and the cylindrical casing, wherein the helical compression spring is configured to compress against the cylindrical casing as pressure is applied to the rod.

19. The method of claim 18, further comprising:
disposing a set of scale marking on an exterior of the cylindrical casing where a slit is formed in the casing; and
disposing an adjustable marker disposed on the exterior of the cylindrical casing, wherein the adjustable marker is configured to be moved along an exterior of the cylindrical casing for calibration of movement of the piston.

20. The method of claim 18, wherein a target compression of the rod and piston when applied to a patient is determined by correlating a force applied to the rod with a compression distance of the helical compression spring.
